(19) United States
(12) Patent Application Publication
(10) Pub. No.: US 2006/0212094 A1
(43) Pub. Date: Sep. 21, 2006

(54) Middle ear multi-channel electrode

(76) Inventors: Ludwig Moser, Estenfeld (DE); Jan Helms, Wuerzburg/Oberduerrbach (DE); Konrad Schwager, Wuerzburg (DE); Claude Jolly, Innsbruck (AT)

Correspondence Address: BROMBERG & SUNSTEIN LLP, 125 SUMMER STREET, BOSTON, MA, 02110-1618, US

(21) Appl. No.: 11/324,014

(22) Filed: Dec. 30, 2005

Related U.S. Application Data
(60) Provisional application No. 60/641,037, filed on Dec. 31, 2004.

(51) Int. Cl. A61N 1/18

(52) U.S. Cl. 607/57

(57) ABSTRACT

An electrode device is for implantation in a human ear. An implantable electrode carrier has one or more electrical contacts on its surface. The electrode carrier and the electrode contacts are adapted to interact with endosteum tissue when implanted in a human ear so as to develop current flow from the electrical contacts through the endosteum tissue toward sensory epithelium within the cochlea.

The present application claims priority from U.S. Provisional Patent Application 60/641,037, filed Dec. 31, 2004, the contents of which are incorporated herein by reference.

### FIELD OF THE INVENTION

The present invention relates to stimulating nerve fibers, and in particular, to a multi-channel electrode for stimulating the middle ear.

### BACKGROUND ART

There are a variety of inner ear conditions and deficits that can benefit from electrical stimulation with single or multi-channel electrodes. Tinnitus, for example, can be masked by electrical stimulation with proper stimulation parameters. Some vestibular dysfunction can be balanced with stimulation of sensory epithelium located in the semicircular canal of the ear. Sensory hearing loss is another major condition that can be compensated with the electrical stimulation of auditory nerve fibers.

High frequency hearing loss is a common condition, especially among the elderly and people afflicted by presbyacusis. Hearing aids are limited in restoring a sense of hearing in the high or mid frequency region. Some patients have losses to the transducer in the inner ear, and depletion of dendrites may have taken place. Currently, hearing aids do not restore high frequency or middle sound sensation for such patients.

Electrical stimulation of the sensory epithelium in the cochlea has been successful with respect to restoring sound sensation for patients deprived of the sound mechanical wave transduction to neural signals to the brain. Since electrical stimulation is successful and surgical techniques have been mastered to enter the middle ear for cochlear implantation, it is now conceivable to use the electrical stimulation for presbyacusis patients, patients afflicted with tinnitus, and other vestibular disorders. Electrical stimulation in combination with auditory amplification may be useful in rehabilitating patients with high frequency hearing loss, as well as patients with sensory hearing loss in a specific frequency bandwidth (including high, low, or mid frequency bandwidth).

However a potential problem with entering the cochlear scalae for stimulating auditory nerve fibers is the potential loss of all residual hearing. It is also not feasible to enter the semi-circular canal for electrical stimulation purposes.

FIG. 1 shows a section view of an ear with a typical cochlear implant system. A normal ear transmits sounds through the outer ear 10 to the eardrum 12, which moves the bones of the middle ear 14, which in turn excites the cochlea 16. The cochlea 16 includes an upper channel, known as the scala vestibuli 18, and a lower channel, known as the scala tympani 20, which are connected by the cochlear duct 22. In response to received sounds transmitted by the middle ear 14, the fluid filled scala vestibuli 18 and scala tympani 20 transmit waves, functioning as a transducer to generate electric pulses that are transmitted to the cochlear nerve 24, and ultimately to the brain.

To overcome total sensorineural hearing loss, a cochlear implant system produces direct electrical stimulation of the cochlea 16. A typical system may include an external microphone that provides an audio signal input to a signal processing stage (not shown) where various signal processing schemes can be implemented. For example, signal processing approaches that are well-known in the field of cochlear implants include continuous interleaved sampling (CIS) digital signal processing, channel specific sampling sequences (CSSS) digital signal processing (as described in U.S. Pat. No. 6,348,070, which is incorporated herein by reference), spectral peak (SPEAK) digital signal processing, and compressed analog (CA) signal processing. Typically, the processed signal is then converted into a digital data format, such as a sequence of data frames, for transmission into an implanted receiver 39.

### SUMMARY OF THE INVENTION

A representative embodiment of the present invention includes an electrode device for implantation in a human ear, and corresponding methods. An implantable electrode carrier has one or more electrical contacts on its surface. The electrode carrier and the plurality of electrode contacts are adapted to interact with endosteum tissue when implanted in a human ear so as to develop current flow from the plurality of electrical contacts through the endosteum tissue toward sensory epithelium within the cochlea.

In further embodiments, an insulator is adapted to restrict current flow toward the outside of the cochlea. The insulator may be separate from the electrode carrier, or an integral part of the electrode carrier. The electrode carrier may be adapted to be positioned in a carrier groove in a bony structure of the inner ear. An adhesive such as fibrin glue may hold the electrode carrier in position in the carrier groove, or the device may be adapted to maintain the electrode carrier in the carrier groove by utilizing hydrostatic forces.

Embodiments also include a cochlear implant system adapted to use an electrode device according to any of the above.

### BRIEF DESCRIPTION OF THE DRAWINGS

FIG. 1 shows a section view of an ear with a typical cochlear implant system.

FIG. 2 shows an electrode with insulating backing in a groove drilled on bony structure of the inner ear in accordance with an embodiment of the invention.

FIG. 3 shows a flow chart for an embodiment which stimulates the high to mod-frequency sound range in the inner ear according to an embodiment of the present invention.

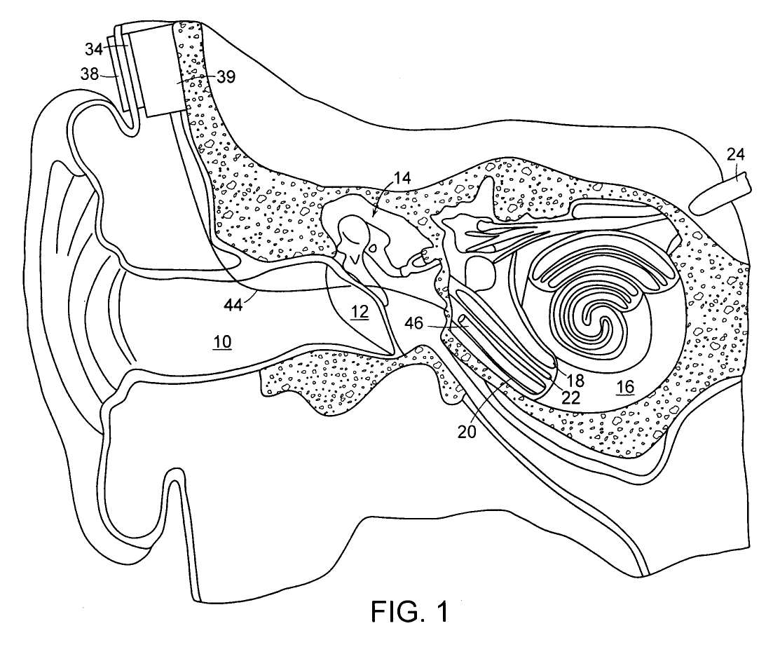

### DETAILED DESCRIPTION OF SPECIFIC EMBODIMENTS

In embodiments of the present invention, a middle ear multi-channel electrode carrier connects to an implantable stimulator module, which together deliver stimulation current to neural tissue of the inner ear. It is possible to create a small channel of a specific length on the outer surface of the bone enclosing the chambers of the inner ear. With the drilled channel close to the membranes lining the inner ear chambers, it is possible to electrically stimulate the sensory epithelium of the inner ear which is located at a close distance from the electrical contacts.

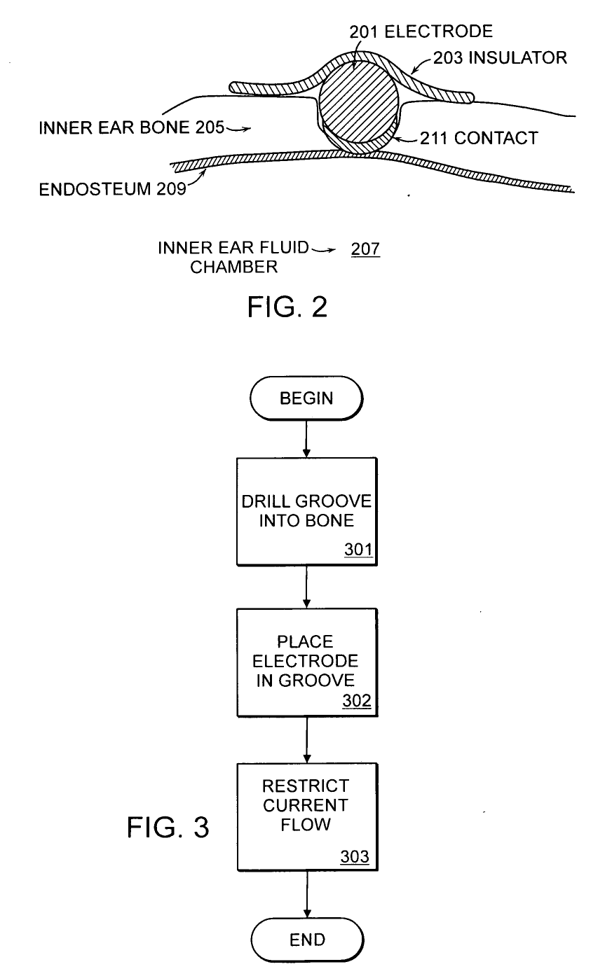

FIG. 2 is an illustration of an electrode carrier 201 placed in a carrier groove 202 drilled on bony structure such as the promontory bone 205 of the inner ear in accordance with embodiments of the invention. The carrier groove 202 may extend down toward the endosteum 209 near the sensory epithelium which lines the internal aspect of the inner ear scalae. The groove 202 drilled into the promontory bone 205 may somewhat follow the direction from base toward apex of the cochlea for a specific length that is tailored to the patient audiogram. The electrode carrier 201 may be snapped or placed into the carrier groove 202.

One or more electrode contacts 211 on the surface of the electrode carrier 201 stimulate the high to mid frequency region of the inner ear without penetrating into the fluid filled chamber 207 of the cochlea. By placing the electrode contacts 211 of the electrode carrier 201 close to the sensory epithelium, stimulation can restore sensations and/or correct other deficiencies of the inner ear.

In one specific embodiment, a biocompatible insulator 203 or insulating backing may include one or more leaves (flat sheets) attached to the electrode carrier 201 on a surface opposite the surface containing the one or more contacts 211. The insulator 203 is designed to restrict current flow to the outside of the cochlea and to promote current flow through the endosteum 209 toward the sensory epithelium, without causing current flow in conductive fluid of the inner ear (which could cause depolarization of the neural tissue). The leaves of the insulator 203 can be glued to the promontory bone 205 to fix in place the electrode carrier 201 (in addition to preventing current flow).

In another embodiment of the invention, the insulator 203 may be separate and not attached to the electrode carrier 201. The electrode carrier 201 may be snapped in place in the drilled groove 202, and the insulator 203 can be placed on the surface of the electrode carrier 201 and effectively maintained in place by hydrostatic forces. Glue, such as fibrin glue, can then be applied to the insulator 203. In yet another embodiment, the electrode carrier 201 is snapped into the drilled groove 202 and no insulating leaf is applied. Glue such as fibrin glue can then be applied to the electrode carrier 201 if necessary.

FIG. 3 shows a flow chart for implantation and use of an embodiment which stimulates the high to mod-frequency sound range in the inner ear. First, a carrier groove is drilled into the surface of the promontory bone of a subject, step 301. Then, an electrode carrier containing one or more electrode contacts is placed in the carrier groove, step 302. The electrode carrier is positioned so that the one or more electrode contacts face the endosteum. Current flow to the outside of the cochlea is restricted, step 303, such that current from the one or more electrode contacts flows toward the sensory epithelium of the cochlea. Restricting current flow may utilize an insulator or insulating backing positioned on or attached to a surface of the electrode carrier.

Entering the inner chamber of the cochlea through a cochleostomy to stimulate neural tissue may cause the patient to lose hearing through disruption of the inner hair cell receptors. One advantage the embodiments described above is that they can provide a multi-channel electrode to stimulate sensory epithelium of the inner ear without entering the chambers of the inner ear. By drilling a carrier groove down toward the sensory epithelium of the scalae, without penetrating the endosteum, an electrode carrier containing one or more electrode contacts can be placed in the carrier groove without entering the inner space of the inner ear. Stimulation current of sufficient intensity can cross the endosteum and stimulate neural tissue located close to or at some distance from the electrode contact.

Although various exemplary embodiments of the invention have been disclosed, it should be apparent to those skilled in the art that various changes and modifications can be made which will achieve some of the advantages of the invention without departing from the true scope of the invention.

What is claimed is:

1. An electrode device for implantation in a human ear, the device comprising:
an implantable electrode carrier; and
a plurality of electrical contacts on a surface of the electrode carrier;
wherein the electrode carrier and the plurality of electrode contacts are adapted to interact with endosteum tissue when implanted in a human ear so as to develop current flow from the plurality of electrical contacts through the endosteum tissue toward sensory epithelium within the cochlea.

2. An electrode device according to claim 1, further comprising:
an insulator adapted to restrict current flow toward the outside of the cochlea.

3. An electrode device according to claim 2, wherein the insulator is separate from the electrode carrier.

4. An electrode device according to claim 2, wherein the insulator is an integral part of the electrode carrier.

5. An electrode device according to claim 1, wherein the electrode carrier is adapted to be positioned in a carrier groove in a bony structure of the inner ear.

6. An electrode device according to claim 5, further comprising:
an adhesive holding the electrode carrier in position in the carrier groove.

7. An electrode device according to claim 6, wherein the adhesive is fibrin glue.

8. An electrode device according to claim 5, wherein the device is adapted to maintain the electrode carrier in the carrier groove by utilizing hydrostatic forces.

9. A cochlear implant system adapted to use an electrode device according to any of claims 1-8.
